(19) United States
(12) Patent Application Publication
(10) Pub. No.: US 2006/0167322 A1
(43) Pub. Date: Jul. 27, 2006

(54) Method for producing menthol

(76) Inventors: Walter Kuhn, Hebbelstr. (DE); Hans-Ulrich Funk, Lindenstr. (DE); Gerhard Senft, Brahmsweg (DE); Konrad A. Korber, Steige (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/525,050

(22) PCT Filed: Aug. 20, 2003
(86) PCT No.: PCT/EP03/09193

§ 371 Date: Feb. 18, 2005

(30) Foreign Application Priority Data
Aug. 22, 2002 (DE) 102 39 274.9

(51) Int. Cl. C07C 35/12

(52) U.S. Cl. 568/830

(57) ABSTRACT

The present invention relates to a method for manufacturing menthol by catalytic hydration of compounds having the carbon network of menthane with at least one double bond and which are substituted in 3-position by oxygen and/or catalytic rearrangement of stereoisomers of the menthol in the presence of hydrogen and doped nickel catalysts.

The present invention relates to a method for manufacturing menthol by catalytic hydration of compounds having the carbon network of menthane with at least one double bond and which are substituted in 3-position by oxygen and/or catalytic rearrangement of stereoisomers of the menthol in the presence of hydrogen and doped nickel catalysts.

Of the naturally occurring cyclical terpene alcohols 1-menthol is a particularly important example. L-menthol has a uniquely refreshing taste, a minty smell and a strong cooling effect on the skin and mucous membrane. It is used in oral hygiene, in cosmetic and pharmaceutical preparations, in tobacco and in confectionery.

The 8 stereoisomers of menthol (two each of enantiomers of menthol, neomenthol, isomenthol and neoisomenthol, see also K. Bauer, D. Garbe and H. Surburg, Common Fragrance and Flavor Materials, 4^{th} Ed., Wiley-VCH, Weinheim 2001, p.p. 52-55) differ according to their organoleptic characteristics. Thus 1-menthol has a characteristic peppermint taste and the abovementioned refreshing effect; it is thus the most valuable menthol stereoisomer. Efforts have therefore been made to perform the hydration in such a way that the greatest possible amount of menthol results or to rearrange stereoismers of the menthol, like those resulting from thymol hydration for example, as effectively as possible into menthol.

The catalytic hydration of compounds having the carbon network of menthane with at least one C═C-double bond and which are substituted in the 3-position by oxygen and/or catalytic rearrangement of stereoisomers of the menthol in the presence of hydrogen leads to racemic menthol.

In Liebigs Annals 1960, 637, 1 the isomerisation of hydrated thymol by means of Raney nickel in methanol at 200° C.-250° C. is described, whereby the product mixture has a content of d,l-menthol of 57.1%.

In EP-A 563 611 and DE-A 197 18 116 menthol is manufactured by catalytic hydration and/or catalytic rearrangement in the presence of hydrogen and noble metal catalysts.

In DE-OS 2 314 813 d,l-menthol is obtained using a Co—Mn-fixed bed catalyst. In a comparative example a mixture which essentially comprised thymol and d-neomenthol, was converted in the presence of Raney nickel at 210° C. and 280 bar hydrogen pressure into d,l-menthol. The reaction product contained 56.1% menthol, and also 4.8% hydrocarbons.

From the commercial point of view the catalytic hydration and/or rearrangement methods known to date produce the menthol only with unsatisfactory chemical yield or selectivity (e.g. menthol content too low, formation of hydrocarbons as undesirable by-products), inadequate space-time-yields (e.g. long reaction times) and/or require expensive noble metal catalysts.

The object of the present invention was to find an improved method for manufacturing menthol by catalytic hydration and/or catalytic rearrangement of menthol, in particular from the commercial aspects and on an industrial scale.

The present invention relates to a method for manufacturing menthol by catalytic hydration of starting materials having the carbon network of menthane with at least one double bond and which are substituted in 3-position by oxygen and/or catalytic rearrangement of stereoisomers of the menthol in the presence of hydrogen, characterised in that the reaction is performed in the presence of a nickel catalyst doped with iron and/or chromium and at a temperature in the range 80-230° C. and hydrogen pressures in the range 1-200 bar abs.

The nickel catalyst in the present invention is a catalyst with a nickel content of at least 50% by weight in relation to the dry catalyst.

Suitable starting materials for the reaction in accordance with the invention are compounds which derive from p-menthane (4-isopropyl-1-methyl-cyclohexane) and are substituted in 3-position by oxygen, such as for example menthone, menthenone, isomenthone, neomenthol, isomenthol, neoisomenthol, isopulegol, neo-isopulegol, iso-isopulegol, neoiso-isopulegol, piperitone, piperitol, piperitenol or isopiperitenol. These compounds can be used in unadulterated enantiomer, enantiomer enriched or racemic form. Thymol, d-menthol and 1-menthol, in particular, can also be used. These compounds can be used both individually and in any mixture as starting materials.

For the method in accordance with the invention, the catalyst can be used in the dry or moist state (water content up to 60% by weight).

The metal contents indicated in the following refer to the weight of the dry catalyst.

Advantageous nickel catalysts within the context of the invention are iron-doped or chromium-doped nickel catalysts, the iron or chromium content in the catalyst of which are 0.1-20% by weight, preferably 1-10% by weight, in each case.

For the method in accordance with the invention, particular preference is for the use of catalysts containing nickel, iron and chromium, which are advantageously in the following proportions by weight:

Fe: 0.1-20% by weight, preferably 1-10% by weight,

Cr: 0.1-20% by weight, preferably 1-10% by weight,

Ni: 60-95% by weight, preferably 80-93% by weight,

and as necessary other metals such as aluminium, silicon, magnesium or zinc.

Preference according to the invention is for the use of Fe- and Cr-doped Raney nickel catalysts (Raney nickel—iron—chromium). Here catalysts containing or comprising the following are particularly advantageous:

Fe: 0.1-20% by weight, with particular preference for 1-10% by weight;

Cr: 0.1-20% by weight, with particular preference for 1-10% by weight,

Ni: 60-95% by weight, with particular preference for 80-93% by weight,

Al: 1-20% by weight, with particular preference for 3-10% by weight.

For the method in accordance with the invention, the ratio of the weight of dry nickel catalyst used to the starting material is 0.001-0.1:1, preferably 0.005-0.05:1, with particular preference for 0.01-0.03:1.

In the case of a continuous method this weight ratio refers to the total quantity of the starting material introduced into the hydration in relation to the total quantity of the nickel catalyst used.

In accordance with the invention, the method is performed at 80-230° C., preferably at 120-210° C. and with particular preference for 150-190° C.

The method in accordance with the invention is performed with hydrogen, with the hydrogen pressure normally being in the range 1 to 200 bar abs.

When the reaction is performed discontinuously hydrogen pressures in the range 1 to 100 bar abs, in particular in the range 3 to 50 bar abs., and in particular in the range 5 to 25 bar abs. are preferred.

When the reaction is performed continuously hydrogen pressures in the range 50 to 200 bar abs, in particular in the range 100 to 200 bar abs., are preferred.

The reaction time is normally in the range 1 to 100 hours, preferably in the range 10 to 70 hours, with particular preference for 20 to 55 hours.

The method can be performed continuously, semi-continuously and discontinuously.

Where the reaction is discontinuous (batch) the doped nickel catalyst is preferably used in the form of powder, advantageously without base material.

Where the reaction is continuous it can be advantageous to use the doped nickel catalyst as a moulding or hollow body. Bodies of any design can be used, such as hollow strands, extrudates, pellets, extruded strands, vortex strands, saddles, rings, hollow spheres, spheres, hollow cylinders, cylinders, cubes, tablets, cones and the like. Here it is advantageous not to use a base material, but to design the doped nickel catalyst as such as a body.

It is similarly advantageous in the continuous method to perform the reaction in a fixed bed reactor, preferably in a fluidised bed reactor.

Table 1 shows the results of the conversion (reaction conditions: 20 bar hydrogen pressure, 175° C., no diluent, 1% by weight dry catalyst, with reference to thymol, water content of the catalyst: 50% by weight) of thymol to various nickel catalysts after 4 hours.

TABLE 1

| | | | Iso- | Neo- | |
|---|---|---|---|---|---|
| | Thymol | Menthol | menthol | menthol | Neoisomenthol |
| Catalyst | GC-% | GC-% | GC-% | GC-% | GC-% |
| Ra—Ni | 42.1 | 1.9 | 12.4 | 4.1 | 19.7 |
| Ra—Ni—Cr | 19.3 | 11.8 | 22.8 | 13.1 | 21.5 |
| Ra—Ni—Fe | 30.8 | 4.3 | 37.7 | 7.4 | 14.5 |
| Ra—Ni—Cr—Fe | 0.1 | 27.3 | 38.5 | 18.5 | 11.6 |

Menthone and isomenthone essentially account for the remaining GC percentages. The catalysts used had the following compositions, the figures refer to the weight of the dry catalyst (source: Degussa AG):

Ra—Ni—Fe: 82% nickel; 7% aluminium; 11% iron

Ra—Ni—Cr: 88% nickel; 9% aluminium; 3% chromium

Ra—Ni—Cr—Fe: 90% nickel; 7% aluminium; 1% iron; 2% chromium

The method in accordance with the invention can be performed using diluents or mixtures of diluents. Alcohols, aqueous alcohols, cyclical or acyclical ether and saturated cyclical or acyclical hydrocarbons are, for example, suitable. Normally diluents such as methanol, ethanol, isopropanol, n-propanol, isobutanol, n-butanol, sec.-butanol, tetrahydrofurane, dibutylether, ethyleneglycoldimethylether, pentane, hexane, heptane, octane, isooctane, cyclopentane, cyclohexane, methylcyclohexane or cyclooctane can be used.

The method in accordance with the invention is preferably performed essentially without diluent. This means a proportion of less than 5% by weight, preferably less than 2% by weight, of diluent with reference to the starting material.

For the method in accordance with the invention additional basic additives from the group comprising alkali hydroxides, alkaline earth hydroxides, alkaline alcoholates, alkaline earth alcoholates and amines can be used.

Of the alkaline hydroxides, sodium hydroxide and potassium hydroxide are preferred. The alkaline and alkaline earth hydroxides can be used in purum or as an aqueous or alcoholic solution.

The alcoholates preferably contain between 1 and 4 carbon atoms and are preferably selected from the group comprising methanolate, ethanolate, isopropanolate and tert.-butanolate, with particular preference for methanolate and ethanolate. Especially preferred alkaline alcoholates are sodium methanolate and sodium ethanolate.

The alcoholates can be used dissolved in purum or in alcohol, preferably here in the alcohol corresponding to the alcoholate. The proportion of alcoholate in alcoholic solution is normally in the range 10-70% by weight, preferably 15-50% by weight.

With the amines triethylamine, tributylamine, ethanolamine and dimethylbenzylamine are preferred.

For the method in accordance with the invention, the ratio of the weight of the basic additive (individually or total of the basic additives) to the starting material is normally 0.000001-0.1:1,preferably 0.00001-0.05:1, with particular preference for 0.001-0.02:1.

The method in accordance with the invention can, by way of example, be performed as follows:

The starting materials, nickel catalyst and, as appropriate, diluent and basic additive, are place in a pressure vessel. The hydration or rearrangement then takes place at the chosen temperature and the chosen hydrogen pressure. Once the reaction is complete the raw menthol is obtained by removing the catalyst (e.g. by filtration, decantation, centrifuging) and if necessary removal of the basic additive through washing. Then the raw menthol is normally further purified, for example by distillation or crystallisation.

When unadulterated enantiomer or enantiomer enriched starting materials are used in the rearrangement reaction racemization or partial racemization takes place under the reaction conditions. The hydrations, racemizations and isomerisations that take place during the method in accordance with the invention surprisingly lead to a large extent to the formation of menthol using an inexpensive nickel catalyst, which is neither based on noble metals nor requires noble metal doping. Particularly surprising is the fact that with the method in accordance with the invention a to a large extent hydrocarbon-free product (typically <0.5% by weight) is obtained.

In particular, the moderate reaction conditions represent a considerable improvement at the industrial level.

The following examples explain the invention:

Unless otherwise stated all data relate to the weight.

### EXAMPLE 1

### Manufacture of d,l-Menthol from Thymol

996 g of thymol (purity: 99.8%) and 20 g Raney-nickel-iron-chromium (composition of the dry catalyst: 92% nickel, 5% aluminium, 2% chromium, 1% iron; water content 50%; form of catalyst: powder; source DegussaAG) are placed in a 5 l agitating autoclave with a gas injection stirrer. Hydration is performed for 24 hours at 175° C. and for 1 hour at 100° C. The hydrogen pressure is 20 bar. Following filtration 1,028 g of a menthol-isomers mixture with the following composition (total menthol 99.5%) are obtained:

60.3% menthol

25.8% neomenthol

11.7% isomenthol

1.7% neoisomenthol

The menthol-isomers mixture obtained can be distilled without organic residue at up to 100° C. sump temperature and 1 mbar.

### EXAMPLE 2

### Manufacture of Menthol from Thymol

The reaction is performed according to Example 1, with the reaction temperature being reduced to 165° C. The menthol-isomers mixture obtained has the following composition (total menthol 99.7%):

60.6% menthol

26.3% neomenthol

11.3% isomenthol

1.5% neoisomenthol

### EXAMPLE 3

### Manufacture of Menthol from Thymol

The reaction is performed according to Example 1, with the reaction temperature being changed to 185° C. The menthol-isomers mixture obtained has the following composition (total menthol 97.7%):

57.6% menthol

26.5% neomenthol

11.7% isomenthol

1.9% neoisomenthol

### EXAMPLE 4

### Manufacture of Menthol from a Menthone-Isomenthone Mixture

996 g of menthone-isomenthone mixture (purity: 99.9%; menthone content 85.8%, isomenthone content 14.2%) and 20 g Raney-nickel-iron-chromium (for composition see example 1) are placed in a 5 l agitating autoclave with a gas injection stirrer. Hydration is performed for 25 hours at 165° C. and for 1 hour at 100° C. The hydrogen pressure is 18 bar. Following filtration 1,003 g of a menthol-isomers mixture with the following composition (total menthol 99.8%) are obtained:

61.9% menthol

25.5% neomenthol

11.1% isomenthol

1.3% neoisomenthol

The menthol-isomers mixture obtained can be distilled without organic residue at up to 100° C. sump temperature and 1 mbar.

### EXAMPLE 5

### Manufacture of Menthol from a Menthol-Isomers Mixture

992 g of a menthol-isomers mixture with the following composition (total menthol 95.3%):

9.2% menthol

81.9% neomenthol

0.1% isomenthol

4.1% neoisomenthol

and 20 g Raney-nickel-iron-chromium (composition: see example 1) are placed in a 51 agitating autoclave with a gas injection stirrer. Isomerisation is performed for 20 hours at 175° C. and for 1 hour at 100° C. The hydrogen pressure is 18 bar. Following filtration 990 g of a menthol-isomers mixture with the following composition (total menthol 94.2%) are obtained:

56.8% menthol

25.0% neomenthol

10.8% isomenthol and

1.6% neoisomenthol

The menthol-isomers mixture obtained can be distilled without organic residue at up to 100° C. sump temperature and 1 mbar vacuum.

### EXAMPLE 6

### Manufacture of Menthol from Isopulegol

993 g of isopulegol with the following composition (total isopulegols 97.6%)

70.1% isopulegol

18.1% neo-isopulegol

6.8% iso-isopulegol and

2.6% neoiso-isopulegol

and 20 g Raney-nickel-iron-chromium (composition: see example 1) are placed in a 5 l agitating autoclave with a gas injection stirrer. Hydration is performed for a total of 20 hours at 175° C. and for 1 hour at 100° C. The hydrogen pressure is 18 bar. Following filtration 1,019 g of a menthol-isomers mixture with the following composition (total menthol 97.0%) are obtained:

61.4% menthol

25.5% neomenthol

8.8% isomenthol

1.3% neoisomenthol

The menthol-isomers mixture obtained can be distilled without organic residue at up to 100° C. sump temperature and 1 mbar.

### EXAMPLE 7

### Manufacture of d,l-Menthol from d-Menthol

985 g of d-menthol (purity: 99.9%; d-menthol >95%, 1-menthol <5%) and 20 g Raney-nickel-iron-chromium (for composition see example 1) are placed in a 5 l agitating autoclave with a gas injection stirrer. The reaction mixture is maintained for 50 hours at 175° C. and for 2 hours at 100° C. at a hydrogen pressure of 18 bar. Following filtration 976g of a menthol-isomers mixture with the following composition (total menthol 99.5%) are obtained:

60.6% d,l-menthol (d-menthol 49%, 1-menthol 51%)

25.8% d,l-neomenthol (d-neomenthol 46%, 1-neomenthol 54%)

11.5% d,l-isomenthol (d-isomenthol 46%, 1-isomenthol 54%)

1.6% d,l-neoisomenthol.

The menthol-isomers mixture obtained can be distilled without organic residue at up to 100° C. sump temperature and 1 mbar vacuum.

The composition is determined by means of GC. GC column for enantiomer separation of the menthol-isomers mixture: pernethylated-β-cyclodextrin from J&W; conditions: carrier gas: helium, 1 bar; temperature programme: 80-180° C., heating rate 2° C./min.

1. Method for manufacturing menthol by catalytic hydration of starting materials having the carbon network of menthane w%ith at least one double bond and which are substituted in 3-position by oxygen and/or catalytic rearrangement of stereoisomers of the menthol in the presence of hydrogen, characterised in that the reaction is performed in the presence of a nickel catalyst doped with iron and/or chromium and at a temperature in the range 80-230° C. and hydrogen pressures in the range 1-200 bar abs., whereby the doped nickel catalyst in the dry state has an iron content of 0.1-20% by weight, a chromium content of 0.1-20% by weight, a nickel content of 60-95% by weight and an aluminum content of 1-20% by weight.

2. Method according to claim 1, characterised in that the nickel catalyst has an iron content of 0.1-10% by weight, a chromium content of 0.1-10% by weight, a nickel content of 80-93% by weight and an aluminum content of 3-10% by weight.

3. Method according to claim 1, characterised in that the method is essentially performed without diluent.

4. Method according to claim 1, characterised in that the nickel catalyst in the dry state has an iron content of 0.1-10% by weight, a chromium content of 0.1-10% by weight, a nickel content of 80-93% by weight and an aluminium content of 3-10% by weight.

5. Method according to claim 1, characterised in that the reaction temperature is between 120 and 210° C.

6. Method according to claim 1, characterised in that the method is performed discontinuously.

7. (canceled)

8. (canceled)

9. Method according to claim 6, characterised in that the hydrogen pressure is between 3 and 50 bar abs.
